(19) United States
(12) Patent Application Publication
(10) Pub. No.: US 2008/0233594 A1
(43) Pub. Date: Sep. 25, 2008

(54) Method For Detecting An At Least Bivalent Analyte Using Two Affinity Reactants

(75) Inventor: Mats Inganas, Uppsala (SE)

Correspondence Address: FULBRIGHT & JAWORSKI, LLP, 1301 MCKINNEY, SUITE 5100, HOUSTON, TX, 77010-3095, US

(73) Assignee: GYROS Patent AB, Uuppsala (SE)

(21) Appl. No.: 11/815,955

(22) PCT Filed: Jan. 17, 2006
(86) PCT No.: PCT/SE2006/000071

§ 371 Date: Jan. 16, 2008

(30) Foreign Application Priority Data
Jan. 17, 2005 (SE) 0500131-8
Jan. 20, 2005 (SE) 11/038712
May 27, 2005 (SE) 60/685554
May 27, 2005 (SE) 0501223-2
Jun. 17, 2005 (SE) 60/691820
Jul. 18, 2005 (SE) 0501684-5

(51) Int. Cl. G01N 33/53; G01N 33/543

(52) U.S. Cl. 435/7.1; 436/518

(57) ABSTRACT

A method for the determination of an analyte which is a) present in a liquid sample 1 suspected of containing the analyte, and b) at least bivalent with respect to simultaneous affinity binding of at least two binding structures BSs. The method comprises formation of an affinity complex that comprises the analyte and an affinity reactant 1 that is immobilized to a solid phase. The method comprises the steps of: (i) providing a microfluidic flow path that comprises a reaction cavity containing a solid phase to which affinity reactant 1 is immobilized, (ii) providing sample 1 upstream of the cavity and flowing it through the cavity for the formation of the affinity complex under flow conditions, (iii) measuring the amount of complex formed in the solid phase by a) incorporating an analytically detectable and soluble affinity reactant 2 that comprises a binding structure BS into the complex subsequent to step (ii), and b) measuring the amount of affinity reactant 2 incorporated.

### FIELD OF INVENTION

The first aspect of the invention relates to a method for the determination of an analyte in a liquid sample 1 suspected of containing the analyte. The method comprises formation of an immobilized affinity complex that comprises the analyte and an affinity reactant 1 that is immobilized to a solid phase. Affinity reactant 1 exposes a binding structure BS that is capable of affinity binding to the analyte. BS is in the complex affinity bound to BS-binding sites on the analyte. The conditions for formation of the complex are selected such that the complex is formed in an amount that is related to the amount of analyte in sample 1. The complex is measured by the use of an analytically detectable affinity reactant 2 that is incorporated into the affinity complex by affinity binding to the analyte.

The analyte is like an antibody at least bivalent in the sense that it is capable of binding simultaneously to at least two equal epitopes. For immunoassay variants of the invention the analyte thus is an at least bivalent anti-BS antibody (Ab) for which each antigen-binding site is capable of binding to BS (=antigen (Ag)) that is part of affinity reactant 1. The term “analyte” thus preferably comprises native and recombinant full length antibodies, recombinant and native (Fab)₂ fragments etc including various kinds of genetically engineered forms that mimic the at least bivalent antigen-binding ability of native antibodies and their bivalent antigen-binding fragments, but the term also comprises other at least bivalent bioorganic affinity reactants that are to be characterized by an affinity assay according to the invention. Examples of non-antibody analytes that potentially are of interest as analytes in the present invention are are streptavidin, IgG-binding proteins deriving from micro-organisms (protein A, G, etc), human fibronectin etc.

Sample 1 typically derives from an original sample that may be equal to sample 1 or may have been obtained by processing an original sample containing an original analyte such that the amount of analyte in sample 1 is related to the amount of original analyte in the original sample.

Plurality of a particular item, such as microchannel structures in a microfluidic device, affinity reactants or analytes etc, means that there are two or more of the item, such as three or more, four or more, five or more etc. See further below.

The terms “affinity reactant” and “affinity counterpart” contemplates that these reactants have the required specificity.

All patent publications and issued patents cited herein are hereby incorporated in their entirety by reference. This in particular applies to U.S. patents and patent applications and international patent applications designating the US.

### TECHNICAL BACKGROUND AND OBJECTS OF THE INVENTION

The kind of assays defined above is well-known in the scientific and patent literature. The detectable affinity reactant 2 normally used has been capable of binding to a class-, subclass-, or species-specific determinant of an antibody analyte and in a few cases to an antigen-binding site. In general statements it has been suggested to use this kind of assays in flow systems, such as microfluidic systems. See for instance WO 02075312, WO 03018198, WO 04083108, WO 04083109 (all of Gyros AB).

U.S. Pat. No. 6,653,066 (Krutzik) describes a test strip based on a sample absorber pad for detecting anti-HIV antibodies in biological samples. Soluble labelled HIV antigen is disposed in an upstream zone and HIV antigen is immobilized in a downstream zone. A sample suspected of containing anti-HIV antibodies is passed through the two zones whereafter labelled HIV antigen bound to the downstream zone is detected. There is no discussion about how to accomplish solid phases that easily can be adapted to antibody analytes of almost any antibody specificity, affinity, and concentration or how to selectively detect a high-affinity or a low-affinity antibody subpopulation in a mixture containing the whole spectra of affinities.

See also Derwent 1989-303835 (Dainippon Pharm)

There are often problems in reaching desired limits of detection, precisions (coefficient of variation, CV), dynamic ranges, signal to noise levels, recoveries, diagnostic specificities and sensitivities, etc in affinity assays. It is therefore a general desire and goal to provide the kind of assays defined above in formats that facilitate acceptable levels with respect to one, two or more of these characteristics, e.g. a) limits of detection ≦10⁻¹⁶ M, such as ≦10⁻⁹ M or ≦10⁻¹² M or ≦10⁻¹³ M or ≦10⁻¹⁴ M or ≦10⁻¹⁵ M or ≦10⁻¹⁶ M, b) dynamic ranges that are more than two, three, four, five or more orders of magnitude, c) precisions (CV) within ±20%, such as within ±10% or within ±5% or within ±3%, d) recoveries ≧70% such as ≧80% or ≧90% or ≧95% or around 100% or more. This desire is more pronounced for microfluidic assays in which sample volumes in the lower end of the μl-range including the nl-range are used, e.g. ≦20 μl, such as ≦5 000 nl or ≦1 000 nl or ≦500 nl. Samples include analyte samples and reagent samples as well as samples used as diluents or washing.

There is often a desire to o carry out various clinical assays on un-diluted or low-diluted samples, for instance with a dilution factor between 1:100 and 1, such as between 1:10 and 1. This general desire is applicable as a goal for the present inventive affinity assay method.

Upon infection or exposure to exogenous antigens as well as upon attempts to actively induce an immune response for vaccination or for preparing antibodies to be used as reactants in assays, capturing, drugs etc, a humoral immune response is evoked and developed over significant periods of time. A full-fledged immune response for protection against exogenous organisms may take several months to develop in a host, typically a vertebrate such as a mammal, an avian etc. Antibodies that are to be used as reactants in immune assays and/or as immobilized or immobilizable capture reactants impose other quality criteria. The corresponding immune response may need to be interrupted at a certain period of time depending on the particular use of the antibodies. The antibody diversity created in an immune response over time may shift between immunoglobulin (Ig) classes/subclasses but may also evolve on a molecular level, i.e. a process of refining the immune response in terms of epitope specificity and affinity, i.e. binding strength. The latter process is called “affinity maturation”. A humoral immune response is polyclonal in the sense that it comprises a spectrum of antibodies originating from different antibody-producing cells and differing with respect to epitope specificity, Ig-class/subclass, affinity etc.

Currently there are no analytical systems that actively and effectively can differentiate humoral immune responses according to their content of low and/or high affinity antibodies. Assay conditions may sometimes be such that high affinity antibodies are preferentially selected for, but in most cases large sample volumes, long diffusion distances and long incubation times will prevent selection of reactive antibodies according to affinity. Antibody assays often measure antibodies of limited significance for an intended use, for instance as a biological marker (i.e. a diagnostic marker for a disease, an infection and the like), as a reactant in an immune assay, as an immobilized or immobilizable capture reactant etc. In diagnostic antigen specific antibody assays clinically less important antibodies may be “included” in the assay result obscuring clinically important antibodies and affecting diagnostic accuracy negatively. Most assays dedicated for assaying presence of antibodies have difficulties in differentiating negative responses from weak positive ones.

One goal of the invention is to provide the above-mentioned antibody assays in formats facilitating discrimination of a high affinity anti-BS antibody subpopulation from a low affinity anti-BS antibody subpopulation where both kinds of subpopulations are directed towards BS of the immobilized affinity reactant 1. For a polyclonal and a monoclonal anti-BS antibody analyte that is a mixture of individual monoclonal anti-BS antibodies, the terms “high” and “low” primarily means that the two subpopulations should contain anti-BS antibodies of affinities in the uppermost part and lowermost part, respectively, of the affinity range defined by the anti-BS antibody analyte. For a monoclonal anti-BS antibody analyte that comprises a single monoclonal anti-BS antibody the terms indicate binding to a higher extent or to a lower extent, respectively (same conditions) where “to a lower extent” includes no detectable binding. Affinity in this context refer to affinity constants i.e. K_{BS—Ab}=[BS—Ab]/[BS][Ab]. For high affinity antibodies the constant is typically ≧10⁸ L/mole, such as ≧10⁹ L/mole or ≧10¹⁰ L/mole or ≧10¹² L/mole. For low affinity antibodies the constant is typically ≦10⁸ l/mole, such as ≦10⁷ L/mole or ≦10⁶ L/mole or ≦10⁴ L/mole. These affinity constants refer to values obtained by a biosensor (surface plasmon reference) from Biacore (Uppsala, Sweden), i.e. with the antigen immobilized to a dextran-coated gold surface.

The analogous goals and definitions are applicable also to non-antibody analytes.

### FIGURES

FIG. 1. A section of the microfluidic device used in the experimental part. The section contains a subset of microchannel structures.

FIG. 2a-b. Measuring range and inter/intra CD assays. FIG. 2a: Rabbit α-PPV was run three times on the same day. Measuring range is over 4 orders of magnitude and both inter- and intra CD shows good reproducibility. A concentration of 100 on the x-axis corresponds to a dilution factor of 125. FIG. 2b: Plotting CVs ranging from 1.96 to 6.08.

FIG. 3a-b. Reproducibility. FIG. 3a: Four standard curves from five mice pooled and serially diluted. The run was repeated four days on the same instrument. By positioning the standard curves on top of each other, good reproducibility could be demonstrated. A concentration of 100 on the x-axis corresponds to a dilution factor of 125. FIG. 3b: Variations are small with CVs ranging from 3.74 to 6.15.

FIG. 4a-c. Standard curves for three different anti-hIgG monoclonals as analyte and various amounts/densities of hIgG as capturing antigen (affinity reactant 1) on the solid phase. Detecting antigen is fluorophor labeled hIgG (affinity reactant 2).

### THE INVENTION

The present inventor has realized that the above-mentioned goals at least partially can be met in the case a) the formation of the immobilized affinity complex between affinity reactant 1 and an antibody analyte is formed while sample 1 is passing through a reaction cavity containing the solid phase under conditions such that at least one of the antigen-binding sites of an anti-BS antibody analyte is left unoccupied, and b) the measurement of the complex is carried out by utilizing an affinity reactant 2 that comprises a binding structure BS (antigen) that is capable of binding to the antigen-binding site of the anti-BS antibody analyte.

The method according to this aspect is in its most general variant characterized by comprising the steps of:
i) providing a flow path comprising a reaction cavity that contains a solid phase to which affinity reactant 1 is immobilized thereby exposing BS for affinity binding to the analyte,
ii) providing sample 1 at a position upstream of the reaction cavity and flowing sample 1 through the reaction cavity for the formation of the immobilized affinity complex under flow conditions and in a form in which one of the BS-binding sites of the analyte is left unoccupied, preferably with significant amounts of complex formation in the upstream part of the solid phase and insignificant amounts of complex formation in the downstream part of the solid phase,
iii) measuring the complex assembled in the solid phase by the use of an analytically detectable and soluble affinity reactant 2 that comprises a binding structure BS that is capable of binding to a BS-binding structures on the analyte, and
iv) optionally relating the measured value found in step (iii) to the amount of analyte in an original sample from which sample 1 derives.

The binding structure BS of affinity reactant 1 and of affinity reactant 2 may be the same or different as long as the BS structure on both two reactants is capable of binding to a BS-binding site on the analyte, e.g. to an antigen-binding site of an antibody analyte. Solubility in this context includes suspensibility.

Step (i): The Flow Path Including the Reaction cavity (104a-h), the Immobilized Binding Structure BS, and the Solid Phase Material.

The flow path is preferably of the kind that is present in microfluidic devices, i.e. a microchannel structure fabricated in a substrate and defined by a system of microconduits that enables the steps of an assay protocol that are to be carried out in the structure to be carried out therein. Typical microfluidic devices have for instance been described by Gyros AB/Amersham Biosciences (WO 99055827, WO 99058245, WO 02074438, WO 02075312, WO 03018198 (US 20030044322) etc); Tecan/Gamera Bioscience (WO 01087487, WO 01087486, WO 00079285, WO 00078455, WO 00069560, WO 98007019, WO 98053311); Å mic AB (WO 03024597, WO 04104585, WO 03101424 etc) etc. In less preferred variants the flow path may be in the form of tubes connecting various functional units such as the reaction cavity, mixing cavity, valve functions etc. In still other variants the flow path is defined by some kind of bibulous material/porous material through which liquid transport can take place by capillary force and the like. The latter variants include various kinds of conventional test strips.

The flow path is typically in the microformat, i.e. has dimensions and/or is capable of handling liquid volumes of the sizes discussed below for microchannel structures/microfluidic devices.

The reaction cavity is in preferred variants in the microformat.

The reaction cavity (104a-h) is defined as the part of the flow path (101a-h) where the solid phase carrying the binding structure BS is present. The upstream/inlet ends of the reaction cavity and of the BS-solid phase thus coincide. In a similar manner the downstream/outlet ends coincide. The reaction microcavity may be a part of a larger chamber in which other solid phases may be placed upstream or downstream of the solid phase in which BS is present. This other solid phases may differ from the BS-solid phase in being devoid of BS and/or with respect to kind of solid phase material. Each of these other solid phases that are devoid of BS may contain one or more other binding structures, e.g. BS″, BS′″ etc that are capable of binding to other analytes (An″, An′″ etc) and thus define other reaction cavities and affinity reactants (AR1″, AR1′″ etc) in the chamber. The same solid phase/reaction microcavity may also contain a number of different immobilized binding structures (BSs). Solid phases that differ from each other with respect to immobilized BS or combination of different BSs may be physically separated by a solid phase that is devoid of BS, i.e. a dummy solid phase.

The reaction cavity (104a-h) is typically in the microformat, i.e. has at least one cross-sectional dimension that is ≦1 000 μm, such as ≦500 μm or ≦200 μm (depth and/or width) and is then called microcavity. The smallest cross-sectional dimension is typically ≧5 μm such as ≧25 μm or ≧50 μm. The total volume of the reaction cavity is typically in the nL-range, such as ≦5 000 nL, such as 1 000 nL or ≦500 nL ≦100 nL or ≦50 nL or ≦25 nL.

The reaction cavity typically has a length that is within the range of 1-100 000 μm, such as ≧10 μm or ≧50 μm or 100 μm or ≧400 μm, and/or ≦50 000 μm or ≦10 000 μm or ≦5 000 μm or ≦2 500 μm or ≦1 000 μm.

The solid phase may be in the form of porous bed, i.e. a porous monolithic bed or a bed of packed particles that may be porous or non-porous. Alternatively, the solid phase may be an inner wall of a reaction cavity. A monolithic bed may be in the form of a porous membrane or a porous plug.

The term “porous particles” has the same meaning as in WO 02075312 (Gyros AB).

Suitable particles are spherical or spheroidal (beaded), or non-spherical. Appropriate mean diameters for particles are typically found in the interval of 1-100 μm with preference for mean diameters that are ≧5 μm, such as ≧10 μm or ≧15 μm and/or ≦50 μm. Also smaller particles can be used, for instance with mean diameters down to 0.1 μm. Diameters refer to the “hydrodynamic” diameters. Particles to be used may be monodisperse (monosized) or polydisperse (polysized) in the same meaning as in WO 02075312 (Gyros AB).

The base material of a solid phase may be made of inorganic and/or organic material. Typical inorganic materials comprise glass. Typical organic materials comprise organic polymers. Polymeric materials comprise inorganic polymers, such as glass and silicone rubber, and organic polymers of synthetic or biological origin (biopolymers). The term “biopolymer” includes semi-synthetic polymers in which there is a polymer backbone derived from a native biopolymer. Appropriate synthetic organic polymers are typically cross-linked and are often obtained by the polymerisation of monomers comprising polymerisable carbon-carbon double bonds. Examples of suitable monomers are hydroxy alkyl acrylates, for instance 2-hydroxyalkyl acrylates such as 2-hydroxyethyl acrylates, and corresponding methacrylates, acryl amides and methacrylamides, vinyl and styryl ethers, alkene substituted polyhydroxy polymers, styrene, etc. Typical biopolymers in most cases exhibit carbohydrate structure, e.g. agarose, dextran, starch etc.

The term “hydrophilic” in the context of a porous bed contemplates a sufficient wettability of the surfaces of the pores for water to be spread by capillarity all throughout the bed when one end of the bed is in contact with excess water (absorption). The expression also means that the inner surfaces of the bed that is in contact with an aqueous liquid medium during step (ii) shall expose a plurality of polar functional groups which each has a heteroatom selected amongst oxygen and nitrogen, for instance. Appropriate functional groups can be selected amongst hydroxy groups, ethylene oxide groups (—X—[CH₂CH₂O—]ₙ where n is an integer >1 and X is nitrogen or oxygen), amino groups, amide groups, ester groups, carboxy groups, sulphone groups etc. For solid phase materials in particle form this means that at least the outer surfaces of the particles have to exhibit polar functional groups. Similar material, wettabilities and functional groups etc also apply to solid phases in the form of inner walls.

A lowering of the density of BS (antigen for antibody analytes) on the solid phase will typically increase the likelihood for an analyte to bridge between BS on the solid phase and BS on the analytically detectable reactant (affinity reactant 2) and thus also increase the likelihood for a good performance of the assay. Once the distance between BS is sufficiently large in the solid phase further lowering is likely to have minor effects on the bridging. The total amount of BS on the solid phase is typical in molar excess compared to the amount of analyte.

The molar ratio between amount of BS on the solid phase and the amount of analyte in the sample that is to pass the solid phase may be ≧2, such as ≧5 or ≧25 or ≧50 or ≧100 or ≧1 000≧ or ≧10 000 or ≧100 000. In variants of the invention where the analyte comprises subpopulations of antibodies that differ in affinity for BS on the solid phase and one wants to selectively avoid capturing subpopulations at the lower end of the affinity range there may be advantages in using smaller excesses and even deficient amounts of solid phase bound BS. In other words the molar ratio between amount of BS on the solid phase and the amount of analyte may depending on variant be ≦100 000, such as ≦10 000 or ≦1 000 or ≦500 or ≦100 or ≦50 or ≦25 or ≦10 or ≦5 or ≦2 or ≦1 or ≦0.8 or ≦0.5.

Experimental testing is typically required for each particular analyte to determine working binding capacities, density of BS, kind of solid phase etc for obtaining an good performance of the inventive method.

Affinity reactants 1 and/or 2 and in particular their BS parts as well as the analyte may exhibit
a) amino acid structure including protein structure such as peptide structure such as poly and oligopeptide structure, and including mimetics and chemically modified forms of these structures etc; b) carbohydrate structure, including mimetics and chemically modified forms of these structures, etc;
c) nucleotide structure including nucleic acid structure, and mimetics and chemically modified variants of these nucleotide structures, etc;
d) lipid structure such as steroid structure, triglyceride structure, etc, and including mimetics and chemically modified forms of these structures;
e) other structures of organic or bio-organic nature

A number of other structures and substances may also be present in the affinity reactants used, for instance in their BS parts. Such other structures and substances can be illustrated with haptenic/antigenic structures of infectious agents (bacteria, algae, fungi, viruses, prions, moulds, parasites etc), drugs, autoantigens, allergens, synthetic or native immunogens that are capable of provoking humoral immune responses used for diagnostic purposes or for the manufacture of an antigen specific antibody reagent or drug.

The technique for the introduction of BS, on the solid phase is typically according to one or both of two main routes:
a) immobilization of an affinity reactant 1 that comprises BS to the solid phase, and
b) building the affinity reactant 1 stepwise on the solid phase (solid phase synthesis of an immobilised reactant that exhibits BS.

Both routes are commonly known in the field. The linkage to the solid phase material may be via covalent bonds, affinity bonds (for instance biospecific affinity bonds), physical adsorption, electrostatic bonds etc.

Alternative a) typically makes use of an immobilizing pair of two reactive structures (RSₛₚ and RSₐᵣ₁) that are mutually reactive with each other to the formation of a bond that resists undesired cleavage during the processing of the method of the invention. RSₛₚ is pre-introduced on the solid phase material before step (i) or is inherently present on the solid phase material. RSₐᵣ₁ is present on affinity reactant 1. The formation of the immobilizing bond is carried out either before or during step (i).

Covalent immobilization means that the bond resulting from reaction of RSₛₚ with RSₐᵣ₁ leads to the formation of a covalent bond that attaches affinity reactant 1 and thus also BS of affinity reactant 1 to the solid phase: The RSₐᵣ₁ group of affinity reactant 1 is typically selected amongst electrophilic and nucleophilic groups. Examples of groups that may be used are amino groups and other groups comprising substituted or unsubstituted —NH₂, carboxy groups (—COOH/—COO⁻), hydroxy groups, thiol groups, disulfide groups, carbonyl groups (keto, aldehyde), groups containing carbon-carbon double and triple bonds, haloalkyl groups, in particular reactive forms (activated forms) of such groups, e.g. reactive esters, reactive amides or imides, alkene and alkyne groups to which one or more carbonyls are directly attached (α-β unsaturated carbonyls), haloalkyl to which a carbonyl group is directly attacked (α-halo carbonyl) etc. Free radical reactions may also be used for the covalent introduction of binding structures BS on the solid phase.

Immobilization via affinity bonds may utilize an immobilizing affinity pair in which one of the members (immobilized ligand, L=RSₛₚ) is firmly attached to the solid phase material. The other member (immobilizing binder, B=RSₐᵣ₁) is used as a conjugate (immobilizing conjugate) comprising binder B and BS (equal to antigen/hapten for antibody analytes). The pair is typically selected such that it does not interfere with the affinity reaction of the affinity reactant to be immobilized and may thus be useful for the immobilization of a range of affinity reactants for which it is non-interfering. In other words the binder B and the affinity ligand L are both generic. Examples of immobilizing affinity pairs are a) biotin-binding compounds such as streptavidin, avidin, neutravidin, anti-biotin antibodies etc and biotin, b) anti-hapten antibodies and the corresponding haptens or antigens, c) IMAC groups (immobilized metal affinity chelates) and an amino acid sequence containing histidyl and/or cysteinyl and/or phosphorylated amino acid residues (i.e. an IMAC motif), d) anti-species specific antibodies and Igs from the corresponding species, e) class/subclass-specific antibodies and Igs from the corresponding class, f) Igs and microbially derived Ig-binding proteins (or vice versa) etc. A fragment or a derivative that exhibits affinity to the same counterpart as the corresponding intact affinity reactant is member of the same pair as the intact pair, i.e. for immunoglobulins the fragment may comprise species- or class/subclass unique Ig parts. Ig means immunoglobulins in mammals as well as the corresponding proteins in other animals.

The term “conjugate” primarily refers to covalent conjugates, such as chemical conjugates and recombinantly produced conjugates, and comprises at least two moieties bound together typically covalently via a linker. For a recombinant conjugate the linker as well as at least one of the moieties have peptide structure. The term also includes so-called native conjugates, i.e. affinity reactants which each exhibits two binding sites that are spaced apart from each other and with affinity directed towards two different molecular entities. Native conjugates thus includes an antigen which has physically separated antigenic determinants that are different, an antibody which comprises a species and/or class-specific determinant in one part of the molecule and an antigen/hapten-binding site in another part.

Preferred immobilizing affinity pairs (L and B) typically have affinity constants (K_{L—B}=[L][B]/[L—B]) that are at most equal to the corresponding affinity constant for streptavidin and biotin, or ≦10¹ times or ≦10² times or ≦10³ times larger than this latter affinity constant. This typically will mean affinity constants that roughly are ≦10 ³ mole/l, ≦10⁻¹² mole/l, ≦10⁻¹¹ mole/l and ≦10⁻¹⁰ mole/l, respectively. The preference is to select L and B amongst biotin-binding compounds and streptavidin-binding compounds, respectively, or vice versa. These affinity constant ranges refer to values obtained by a biosensor (surface plasmon resonance) from Biacore (Uppsala, Sweden), i.e. with the ligand L immobilized to a dextran-coated gold surface.

The binding capacity of the solid phase for RSₐᵣ₁ can be measured as the amount of RSₛₚ in mole per unit volume, disregard blocking and destruction of binding sites caused by the immobilization. With this measure suitable binding capacities will typically be found within the interval of 0.001-3000 pmole, such as 0.01-300 pmole, divided by nL solid phase in bed form saturated with liquid. For instance, if 0.1 pmole streptavidin per nL has been immobilized this corresponds 0.4 pmole/nL biotin-binding sites. The conversion factor four is because streptavidin has four binding sites for biotin per streptavidin molecule.

Binding capacity can also be measured as actual binding capacity for RSₐᵣ₁, i.e. mole active RSₐᵣ₁ structures per unit volume of the solid phase that contains RSₛₚ (bed form and saturated with liquid such as water). This kind of binding capacity will depend on the immobilization technique, the pore sizes of the solid phase, the size of the entity to be immobilized, the material and design of the solid phase etc. Ideally the same ranges apply for the actual binding capacity as for the total amount of binding sites (as defined above).

Measurements of actual binding capacities can be carried out according to principles well known in the field. This typically means that RSₛₚ of the solid phase is saturated with an excess reagent containing RSₐᵣ₁, whereafter the amount bound to RSₛₚ is measured, for instance directly on the solid phase or after elution. To facilitate measurement, labeled forms of reagents may be used, for instance by the use of a mixture of labeled and unlabelled reagent containing RSₐᵣ₁.

In the case of immobilizing affinity pairs, the actual binding capacity primarily refers to binding/capturing of the binder B in its basic form, e.g. unconjugated and/or underivatized.

In the case the solid phase is an inner wall of the reaction microcavity, the volume of the solid phase is taken as the volume of the reaction microcavity.

The optimal range of binding capacities (for RSₐᵣ₁) for a particular assay depends on a number of factors, e.g. kind of solute and/or the analyte and/or the concentration range in which the solute/analyte is measured, immobilizing pair such as the immobilizing affinity pair used, the kind of solid phase, e.g. porosity and its base material, size of conjugate if an immobilizing affinity pair is used, etc. Testing by trial and error is at the moment the safest way to optimize the binding capacity in relation to a particular assay variant.

A reactive structure RSₛₚ and/or BS may be introduced on the solid phase while the solid phase is placed in a reaction cavity of a flow path or in a batch mode while the solid phase is placed outside the flow path. A portion of the RSₛₚ solid phase material is after the introduction of RSₛₚ in a batch mode transferred to a reaction cavity of a flow path where it may be further transformed to exhibit BS. The latter transformation may also take place in a batch mode outside the flow path. Alternatively both steps are carried out while the solid phase is placed in a reaction cavity of a flow path. It may be important to have a homogeneous distribution of BS in the flow direction or at least a sufficient excess of BS in an upstream part of the solid phase. Appropriate distribution can be accomplished by performing both of the steps in a batch mode followed by a transfer of a portion of the solid phase to the flow path.

Densities and amounts of BS on the solid phase can easily be varied in a controlled manner by carrying out the immobilization of affinity reactant 1 to an activated form of a solid phase containing RSₛₚ in an inhibition mode, such as a competitive mode. In other words contacting an RSₛₚ solid phase with a liquid sample containing affinity reactant 1 in a form exhibiting RSₐᵣ₁ and a nonsense reactant that exhibits a structure RSₙₛ that is reactive with RSₛₚ to give the kind of linkage discussed above. Both of the reactants or the total amount of reactants shall typically be in excess compared to the amount of RSₛₚ groups. The actual density of BS in the final solid phase will then be determined by the relation between the rates of the immobilization reaction for affinity reactant 1 and the nonsense reactant. In this way it will be simple for the customer to design reaction cavities that differ in wide ranges with respect to amount and density of a desired BS. The inhibition introduction of BS may be carried out in a batch mode or with the RSₛₚ solid phase in a reaction cavity and with reaction under flow conditions. As shown in the experimental part this will simplify the construction and designs of solid phases for the quantitation of analyte subpopulations differing in affinity for a given BS.

In an alternative variant the nonsense reactant is reacted with the solid phase before or after affinity reactant 1 has been immobilized. In this variant it is important to secure that at least a fraction of the RSₛₚ remains after the first step.

In certain variants the liquid sample containing affinity reactant 1 and used to introduce BS structures on the solid phase also contains one or more additional affinity reactants each of which has a) a reactive structure RS that is capable of reacting with RSₛₚ as discussed for RSₐᵣ₁ above, and b) affinity for other at least bivalent analytes of the type discussed above. In other words the liquid sample contains an affinity reactant 1² with specificity for a first analyte, an affinity reactant 1² with specificity for a second analyte, an affinity reactant 1³ with specificity for a third analyte etc. In these variants the final solid phase will contain a plurality of affinity reactants and will be useful for assaying two or more analytes in the same liquid sample (multiplexing). Each analyte will require its own detectable affinity reactant 2 with its own unique binding structure BS (2¹, 2², 2³ etc).

In the case an irrelevant reactant is used to neutralize reactive structures RSₛₚ that are not used for introduction of BS, then the final solid phase used in step (ii) will contain structures that derives from RSₛₚ which have not been utilized for the introduction of BS (affinity reactant 1) on the solid phase. These solid phases are part of a separate invention “A separate aspect of the invention” which is described in our copending international application filed in parallel with this application.

Immobilizing affinity pairs are preferred as RSₛₚ and RSₐᵣ₁.

Step (ii): Flow Conditions and Capturing

The term “flow conditions” in step (ii) means that the analyte, such as an anti-BS antibody analyte, is presented to the BS on the solid phase while the liquid in which the analyte is present is continuously flowing through the reaction cavity/solid phase for the period of time during which the affinity complex between the analyte and BS of the solid phase is formed. The flow rate used should preferably provide non-diffusion limiting conditions for the affinity reaction even if also diffusion-limiting conditions may be used. Flow rates providing non-diffusion limiting conditions are characterized in that they result in an enrichment (peak) of captured analyte in an upstream section of the solid phase even if a minor fraction of the analyte may be present in more downstream sections of the solid phase exposing BS structures. An analyte subpopulation, such as an anti-BS antibody analyte subpopulation that is captured in such a peak is typically of higher affinity than an anti-BS antibody analyte subpopulation that is captured downstream of the peak or have passed through the solid phase without being captured.

The appropriate flow rate through the porous bed depends on a number of factors:
a) immobilized affinity reactant 1;
b) the kind of analyte, e.g. kind of antibody analyte including species origin, Ig-class and/or subclass, affinity constant of the analyte of interest;
c) the dimensions of the reaction cavity (volume, length etc),
d) the kind of solid phase (the solid phase material, porosity, bed or coated inner wall etc); and
e) etc.

Typically the flow rate should give a residence time of ≧0.010 seconds such as ≧0.050 sec or ≧0.1 sec for the liquid sample 1, i.e. the liquid aliquot containing the analyte and passing through the reaction cavity. The upper limit for residence time is typically below 2 hours such as below 1 hour. Illustrative flow rates are within 0.001-10 000 nL/sec, such as 0.01-1 000 nL/sec or 0.01-100 nL/sec or 0.1-10 nL/sec. These flow rate intervals may primarily be useful for solid phase volumes in the range of 1-1 000 nL, such as 1-200 nL or 1-50 nL or 1-25 nL. Residence time refers to the time it takes for a liquid aliquot to pass the solid phase in the reaction cavity. Optimization typically will require experimental testing for each particular system.

Flow conditions as defined above are preferably also applied during the capture of other affinity reactants to the solid phase, e.g. affinity reactant 2 (step (iii)). The same general trends for creating flow conditions favouring selective measurement of high affinity analyte subpopulations are likely to apply also for capturing of affinity reactant 2 to the solid phase when taking place as a part of step (iii).

Flow conditions favouring selective determination of high-affinity analyte subpopulations are further discussed below in this specification. See also WO 02075312 (Gyros AB).

The liquid flow through the solid phase can be driven by in principle any kind of forces, e.g. electrokinetically or non-electrokinetically created forces with preference for centrifugal force possibly combined with capillary force for flow paths in microfluidic devices adapted for this. See further below.

Step (iii). Measurement of the Result of Step (ii).

This step means measurement of the amount of anti-BS antibody analyte bound to BS on the solid phase during step (ii) and may include measurement of the distribution of analyte in the flow direction of the solid phase. The measurement is accomplished by the use of affinity reactant 2 that exhibits BS and therefore can be incorporated into the immobilized complex formed in step (ii).

Analytically detectable affinity reactant 2 comprises one moiety 1 in which there is a detectable group and another moiety 2 in which there is a BS that is capable of affinity binding to one of the BS-binding sites of the analyte. Affinity reactant 2 thus is a conjugate in which moiety 1 and moiety 2 are firmly attached to each other, preferably by covalent bonds. This conjugate may be native or synthetic. For synthetic conjugates the detectable group will be called “label” or “tag”.

Binding of affinity reactant 2 can take place either a) before step (ii) or b) during step (iii). BS-binding sites on the analyte that are utilized for binding to affinity reactant 2 are not available for binding to BS on the solid phase.

Alternative (a) means that the analyte is entering the reaction cavity in step (ii) as a pre-formed affinity complex that comprises the analyte and affinity reactant 2 where BS of reactant 2 binds to a BS-binding site of the analyte leaving at least one BS-binding site on the analyte available for affinity capturing by BS on the solid phase (via immobilized affinity reactant 1). The pre-formed affinity complex may have been created outside the flow path or within the flow path at a position upstream of the reaction cavity. The conditions for this preformation are selected such that amount of the pre-formed complex becomes related to the actual amount of anti-BS antibody analyte in an original sample. When sample 1 is passing through the reaction cavity, BS on the solid phase captures the pre-formed affinity complex. The resultant immobilized complex thus comprises the analyte, affinity reactant 2 and affinity reactant 1.

Alternative (b) means that affinity reactant 2 is provided in a separate liquid sample 2 in a position upstream of the reaction cavity. This sample 2 is passed through the reaction cavity subsequent to sample 1. There may be one or more washing steps between sample 1 and sample 2. BS-binding sites that remain unoccupied on the analyte after step (ii) will then capture affinity reactant 2. The ternary immobilized complex will in principle be of the same kind as in alternative (a).

The detectability of moiety 2 of affinity reactant 2 may reside in the fact that the moiety comprises a group that can be analytically detected and quantified. Signal-generating groups and affinity groups are typical examples of useful detectable groups. A signal-generating group may be selected amongst radiation emitting or radiation absorbing groups and groups that in other ways interfere with a given radiation. Particular signal-generating groups are enzymatically active groups including enzymes, cofactors, substrates, coenzymes etc; groups containing particular isotopes such as radioactive or non-radioactive isotopes; fluorescent and fluorogenic groups; luminescent group including chemiluminescent groups, bioluminescent groups etc; metal-containing groups including groups containing a metal ion etc. Affinity groups in this context are typically detected by the use of an affinity reactant 3 that is a conjugate between an affinity counterpart to the detectable affinity group and a second detectable group that is different from the detectable group of affinity reactant 2, and preferably is a signal-generating group typically in the form of a label. Typical affinity based detectable groups may be selected amongst the individual members of the immobilizing affinity pairs discussed above, with the proviso that an affinity based detectable group should not be capable of affinity binding during the method to a member of an immobilizing binding pair if such a pair is used for immobilization of BS to the solid phase.

In other variants affinity reactant 2 is detectable due to the increase in volume or mass the reactant adds to the affinity complex formed on the solid phase. In other words the reactant as such defines the detectable group. See for instance WO 03102559 (Gyros AB).

The actual measurement can be carried out in the reaction microcavity in the case the detectable group and the method for measurement has been so designed. This is particularly important if the distribution of the analyte along the flow direction in the reaction cavity/solid phase is used for determining the affinity between an analyte and BS that has been pre-immobilized to the solid phase. See for instance WO 02075312 (Gyros AB) and PCT/SE2005/001153 (Gyros AB). If only the amount of the analyte captured to the solid phase is desired, the actual measurement may take place downstream of the reaction cavity, for instance in a separate detection cavity. This may involve measurement of excess of affinity reactant 2 that passes through the reaction cavity or of a soluble and detectable entity generated from the complex formed on the solid phase in step (ii). Affinity reactant 1 may for instance be immobilized to the solid phase by a cleavable bond. As an alternative the analytically detectable group in an analytically detectable reactant that is used, e.g. affinity reactant 2, may be bound to other parts of the detectable reactant by a cleavable linker. See for instance U.S. Pat. No. 4,231,999 (Pharmacia Diagnostics AB). After formation of the complex and subsequent washing, if needed, the cleavable linker/bond is splitted, and released fragments containing the detectable group transported downstream to a detection cavity where they are measured. Another alternative is that the detectable group is a reactant in a reaction system that gives rise to a soluble product that can be transported downstream for measurement. Suitable reaction systems enabling the last alternative comprise catalytic systems, such as biocatalytic systems including enzyme systems, in which case the detectable group may be a component of the system, such as a catalyst, a co-catalyst, a co-factor, a substrate, a co-substrate, an inhibitor, an effector etc. For enzyme systems the relevant components are enzyme, coenzyme, co-factor, substrate, co-substrate, inhibitor, activator, effector etc.

A signal-generating group on a detectable affinity reactant that is incorporated in the affinity complex may be combined with a second signal-generating group. This second group may be selected such that the two groups together give the appropriate signal when the complex is formed or dissociated. This variant may be illustrated with scintillation proximity assays (SPA) in which a soluble affinity reactant, such as affinity reactant 2, which is labelled with tritium, is used together with a solid phase comprising a scintillation substance. The principle with interacting labels may also be illustrated with pairs of fluorophores that may be identical or different and with fluorescence-quencher pairs.

In still another variant the immobilized BS is associated with a signal-generating group for which the signal is changed when affinity reactant 2 is bound via the analyte to the solid phase.

Measurement and treatment of obtained signal data are preferably carried out as outlined in WO 03025548, US 20030156763, WO 03056517, U.S. Ser. No. 10/331,399, WO 05001766 (all Gyros AB).

The distribution may be measured as the relative amount of captured analyte in an upper section (upstream section) of the solid phase (e.g. next to the inlet end of the solid phase containing the antigen), for instance encompassing ≦75%, with preference for ≦50% or ≦30% of (a) the length of the solid phase/reaction cavity, or (b) the part of the solid phase/reaction microcavity from which signal over the tresh-hold can be measured. This relative amount may be called “peak”. Determination of distribution may be particularly interesting for antibody analytes, such as an antibody analyte of a humoral immune response,

Step (iv). Quantification.

This step comprises that one relates a measured value found in step (iii) to the amount of analyte in an original sample from which sample 1 derives and/or to the amount of analyte in sample 1. This is typically done according known principles by comparing with values that have been obtained for one or more standard samples. Typically standard samples comprise a) a series of one, two or more samples containing varying known amounts of analytes, b) one or more samples obtained at an earlier occasion for instance from the same or a different individual, c) one or more samples obtained from healthy individuals or from individuals having a particular disease state, etc. The quantification is typically absolute. It may also be relative, e.g. relative to some kind of constituents of sample 1 or of the original sample, relative to another sample taken at an earlier or later occasion and/or from the same or another individual etc.

Certain kinds of analytes, such as a polyclonal antibody analyte, typically contain a spectrum analyte subpopulations directed towards the same binding structures but with different affinities. During step (ii) low affinity analyte subpopulations will have a tendency to escape through the solid phase without being bound. This may be accomplished if the amount of BS (=antigen) on the solid phase has been selected as suggested by our results with three different anti-IgG monoclonals (see the experimental part) and elsewhere in this specification. It may sometimes be of interest to know the amount of high and/or low affinity analyte subpopulations relative to the total amount of the analyte. This typically requires an estimation of the total amount of the analyte or at least of a larger fraction of the analyte. The latter variant may be accomplished by repeating steps (i)-(iv) with a lower flow rate during the affinity capture step (ii) and/or with a solid phase having an increased capacity for binding the analyte. Alternatively a conventional affinity assay for the analyte may be performed during static conditions for a period of time allowing for the capturing reaction to go to equilibrium. A slower flow rate will increase the chances for capturing analyte subpopulations of lower affinity, i.e. the peak will be more distinct if the flow rate is decreased. The alternative with static conditions will measure both low and high affinity analyte subpopulations. In both alternatives the result is likely to enable a fair estimation of the level of low as well as of high affinity analyte subpopulations in the sample.

Miscellaneous

The method of the invention is likely to be of value for the diagnoses of various medicinal states of animals, including humans. Method variants which exclude measurement of low affinity subpopulations of analytes, such as measuring selectively high affinity anti-BS antibody subpopulations of a polyclonal anti-BS antibody analyte, are likely to provide clear improvements. Interesting diseases are autoimmune diseases, parasitic infestations, infectious diseases including infections by bacteria, viruses, fungi, moulds and other diseases for which an antigen-specific antibody assay may be used in the clinical diagnosis of an individual. Either the total amount of anti-BS antibodies or anti-BS antibodies classified as high affinity antibodies are measured and used as an indicator of the disease as such or of the severity of the disease. A distribution of captured antibodies towards the inlet end (upper section), preferably in the form of a distinct peak will then be indicative of higher levels and/or higher affinities of high affinity anti-BS antibodies. For many of the diseases mentioned this will be indicative of a more accurate diagnosis, and/or the severeness and/or the state of the disease. A more distinct peak, for instance in terms of width and height as discussed above, is therefore likely to indicate a larger diagnostic value. This does not exclude that a distribution towards the outlet end (lower section) of the solid phase might also be useful. It may at least be of value as a negative marker indicating that a certain diseased condition is likely not to be severe or not at hand.

One can envisage that for clinical diagnostic assays, the inventive method at least with respect to variants aiming at measuring high-affinity antibody subpopulation potentially will imply an improved diagnostic specificity. The improvement factors ≧1.1, such as ≧1.5 or even more, such as ≧2.0 compared to performing the same assay under diffusion limiting conditions can be envisaged. In this context diffusion limiting conditions typically refer to static conditions (=non-flow conditions) for a sufficient period of time to reach equilibrium in the capture step in which the analyte is bound to BS on the solid phase (corresponds to to flowing in step (ii) above) possibly combined with static conditions also for binding of the analytically detectable affinity reactant 2 to captured analyte (corresponds to flowing in step (iii) above.

Samples

The liquid samples/liquid aliquots transported and processed in the flow path are typically aqueous including diluents, wash liquids and/or liquids containing a reactant such as the analyte and/or a reagent used, such as affinity reactant 2. Liquid sample 1 that contains the analyte may be an unprocessed biological fluid sample or may derive from such a fluid. Processing to obtain liquid sample 1 may take place within and/or outside the flow path. This processing may include a) transforming an original analyte to a form of the analyte as it exists in liquid sample 1, b) diluting, c) removal of cells and/or other particulate material etc. If not otherwise suggested by the particular context, the term “analyte” in this specification contemplates an original analyte as well as any transformed form thereof as long as the amount of transformed analyte is a function of the amount for original analyte in the original sample.

The term “biological fluid” contemplates any fluid that contains a bio-organic compound that exhibits a structure of the kind indicated above for affinity reactants 1 and 2. In a more narrow sense the same term contemplates a fluid which contains this kind of bio-organic compounds and derives from a fluid the composition of which at least partially has been determined by living or dead biological material. Typical biological fluids include cell culture supernatants, tissue homogenates, blood and various blood fractions such as serum or plasma, lachrymal fluid, regurgitated fluid, urine, sweat, semen, cerebrospinal fluid, gastric juice, saliva, lymph, etc as well as various liquid preparations containing a bio-organic compound as discussed above and deriving from these particular biological fluids. For antibody analytes liquid sample 1 typically derives from a vertebrate body fluid of the kinds discussed above that contains antibodies of a humoral immune response provoked in the vertebrate. Typical vertebrates are mammals, avians, amphibians, reptiles etc. Typical mammals are whales, humans, mice, rats, guinea pig, horses, cows, pigs, dogs, cats etc. Typical avians are hens, canaries, budgerigars etc. Amongst amphibians and reptiles may be mentioned those that are used as pets or are popular in zoological gardens.

Preferred Flow Paths (i.e. in Microfluidic Devices)

A microfluidic device is a device that comprises one, two or more microchannel structures (101a-h) in which one or more liquid aliquots/samples, e.g. liquid sample 1 and/or 2, that have volumes in the μL-range, typically in the nanolitre (nL) range, and contains various kinds of reactants, such as analytes and reagents, products, samples, buffers and/or the like are processed. Each microchannel structure (101a-h) comprises all the functional parts needed for performing the steps of the innovative assay that are to be performed within the microfluidic device. The μL-range contemplates volumes ≦1 000 μL, such as ≦100 μL or ≦10 μL and includes the nL-range that has an upper end of 5 000 nL but in most cases relates to volumes ≦1 000 nL, such as ≦500 nL or ≦100 nL. The nL-range includes the picolitre (pL) range. A microchannel structure comprises one or more cavities and/or conduits that have a cross-sectional dimension that is ≦10³ μm, preferably ≦5×10² μm, such as ≦10² μm.

A microchannel structure (101a-h) of the microfluidic device thus may comprise one, two, three or more functional units selected among: a) inlet arrangements (102,103a-h) comprising for instance an inlet port/inlet opening (105a-b,107a-h), possibly together with a volume-metering unit (106a-h,108a-h) (for metering liquid aliquots to be processed within the device), b) microconduits for liquid transport, c) reaction microcavities (104a-h); d) mixing microcavities/units; e) units for separating particulate matters from liquids (may be present in the inlet arrangement), f) units for separating dissolved or suspended components in the sample from each other, for instance by capillary electrophoresis, chromatography and the like; g) detection microcavities; h) waste conduits/microcavities (112,115a-h); i) valves (109a-h,110a-h); j) vents (116a-i) to ambient atmosphere; liquid splits (liquid routers) etc. A functional unit may have several functionalities, e.g. a reaction microcavity (104a-h) and a detection microcavity may coincide. Various kinds of functional units in microfluidic devices have been described by Gyros AB/Amersham Pharmacia Biotech AB: WO 99055827, WO 99058245, WO 02074438, WO 02075312, WO 03018198 (US 20030044322), WO 03034598, WO 05032999 (U.S. Ser. No. 10/957,452), WO 04103890, WO 2005094976 and by Tecan/Gamera Biosciences: WO 01087487, WO 01087486, WO 00079285, WO 00078455, WO 00069560, WO 98007019, WO 98053311.

In advantageous forms a reaction microcavity (104a-h) intended for a hydrophilic porous bed is connected to one or more inlet arrangements (upstream direction) (102,103a-h), each of which comprises an inlet port (105a-b,107a-h) and at least one volume-metering unit (106a-h,108a-h). One advantageous variant of inlet arrangement (103a-h) is only connected to one microchannel structure (101a-h) and/or reaction microcavity (104a-h) intended to contain the solid phase material. Another advantageous inlet arrangement (102) is common to all or a subset (100) of microchannel structures (101a-h) and/or reaction microcavities (104a-h) intended to contain the solid phase material. This latter variant comprises a common inlet port (105a-b) and a distribution manifold with one volume-metering unit (106a-h) for each microchannel structure/reaction microcavity (101a-h/104a-h) of the subset (100). In both variants, each of the volume-metering units (106a-h,108a-h) in turn is communicating with downstream parts of its microchannel structure (101a-h), e.g. the reaction microcavity (104a-h). Microchannel structures linked together by a common inlet arrangement (102) and/or common distribution manifold define a group or subset (100) of microchannel structures. Each volume-metering unit (106a-h,108a-h) typically has a valve (109a-h,110a-h) at its outlet end. This valve is typically passive, for instance utilizing a change in chemical surface characteristics at the outlet end, such as a boundary between a hydrophilic and hydrophobic surface (hydrophobic surface break) (WO 99058245, WO 2004103890, WO 2004103891 and U.S. Ser. No. 10/849,321 (Amersham Pharmacia Biotech AB and Gyros AB)) and/or in geometric/physical surface characteristics (WO 98007019 (Gamera)).

Typical inlet arrangements with inlet ports, volume-metering units, distribution manifolds, valves etc have been presented in WO 02074438, WO 02075312, WO 02075775 and WO 02075776 (all Gyros AB).

In the case the innovative method comprises that an affinity complex between an analyte and affinity reactant 2 is formed within the microchannel structure prior to step (ii) there is a mixing function between the reaction cavity and the inlet(s) for the liquid sample containing the analyte and the liquid sample containing affinity reactant 2. The two liquid aliquots may be introduced via separate inlet ports or via a common inlet port. The mixing function is typically based on
a) mixing in a mixing microconduit, and/or
b) collecting the two liquid samples in a separate mixing microcavity and cause mixing by
   i) a mechanical mixer e.g. by including magnetic particles in the mixing microcavity, and/or
   ii) the use of inertia force possible enforced by including magnetic particles in the microcavity combined with external magnets, and/or
   iii) back and forth transport in a microconduit connected to the mixing microcavity.

Accelerating and/or decelerating movements of the device, for instance by spinning back and forth, may create inertia force. Back and forth transport may be caused by using capillary transport in one direction and centrifugal force in the opposite direction, i.e. intermittent spinning of the appropriate microfluidic device. The outlet of the mixing microcavity is typically equipped with a valve, such as a non-closing valve for instance a passive valve defined by a hydrophobic break or an abrupt change in a lateral cross-dimension of the inner surface/wall of a microconduit of a microchannel structure. One or both of the inlet ports may or may not be equipped with a volume-defining unit as described in the previous paragraph. Mixing as described above has been presented in U.S. Pat. No. 6,582,662 (Tecan); U.S. Pat. No. 6,572,432 (Tecan), US 20020025583 (First Medical Inc), WO 02074438 (Gyros AB), WO 03018198, WO 05032399 (Gyros AB), PCT/SE2005/000403 (Gyros AB), U.S. Pat. No. 5,591,643 (Abaxis), WO 2004103891 (Gyros AB) etc.

Each microchannel structure has at least one inlet opening (105a-b,107a-h) for liquids and at least one outlet opening for excess of air (vents) (116a-i,112) and possibly also for liquids (circles in the waste channel (112)).

The microfluidic device contains a plurality of microchannel structures per device where a single structure is intended to contain the solid phase according to the invention. Plurality in this context means two, three or more microchannel structures and typically is ≧10, e.g. ≧25 or ≧90 or ≧180 or ≧270 or ≧360.

Different principles may be utilized for transporting the liquid within the microfluidic device/microchannel structures between two or more of the functional units. Inertia force may be used, for instance by spinning the disc as discussed in the subsequent paragraph. Other useful forces are capillary forces, electrokinetic forces, non-electrokinetic forces such as capillary forces, hydrostatic pressure etc.

The microfluidic device typically is in the form of a disc. The preferred formats have an axis of symmetry (Cₙ) that is perpendicular to or coincides with the disc plane, where n is an integer ≧2, 3, 4 or 5, preferably ∞ (C_{∞}). In other words the disc may be rectangular, such as square-shaped and other polygonal forms but is preferably circular. Spinning the device around a spin axis that typically is perpendicular or parallel to the disc plane may create the necessary centrifugal force. Variants in which the spin axis is not perpendicular to a disc plane are given in WO 04050247 (Gyros AB).

If centrifugal force is used for driving liquid flow through the reaction microcavity/solid phase, the reaction microcavity is typically oriented with the flow direction essentially radially outwards from the spin axis.

The preferred devices are typically disc-shaped with sizes and/or forms similar to the conventional CD-format, e.g. sizes that are in the interval from 10% up to 300% of a circular disc with the conventional CD-diameter (12 cm).

Microchannels/microcavities of a microfluidic device may be manufactured from an essentially planar substrate surface that exhibits the channels/cavities in uncovered form that in a subsequent step are covered by another essentially planar substrate (lid). See WO 91016966 (Pharmacia Biotech AB) and WO 01054810 (Gyros AB). Both substrates are preferably fabricated from plastic material, e.g. plastic polymeric material.

The fouling activity and hydrophilicity of inner surfaces should be balanced in relation to the application. See for instance WO 0147637 (Gyros AB).

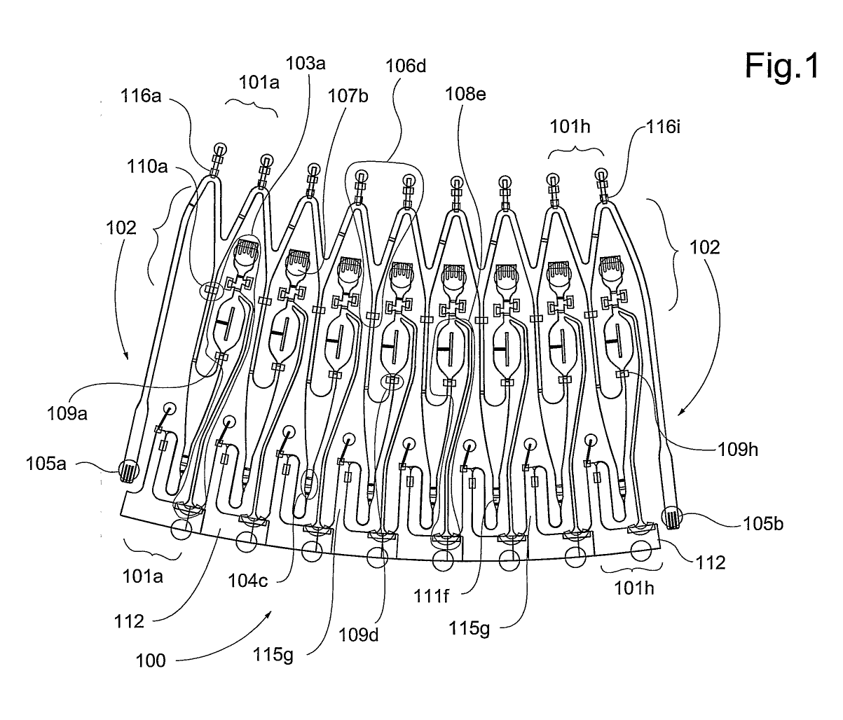

The terms “wettable” (hydrophilic) and “non-wettable” (hydrophobic) in the context of inner surfaces of a microchannel structure contemplate that a surface has a water contact angle ≦90° or ≧90°, respectively. In order to facilitate efficient transport of a liquid between different functional parts, inner surfaces of the individual parts should primarily be wettable, preferably with a contact angle ≦60° such as ≦50° or ≦40° or ≦30° or ≦20°. These wettability values apply for at least one, two, three or four of the inner walls of a microconduit. In the case one or more of the inner walls have a higher water contact angle, for instance is hydrophobic, this can be compensated for by a more wettable surfaces of one or more of the other inner wall(s). The wettability, in particular in inlet arrangements should be adapted such that an aqueous liquid to be used will be able to fill up an intended microcavity/microconduit by capillarity (self suction) once the liquid has started to enter the cavity/microconduit. A hydrophilic inner surface in a microchannel structure may comprise one or more local hydrophobic surface breaks in a hydrophilic inner wall, for instance for introducing a passive valve, an anti-wicking means, a vent solely function as a vent to ambient atmosphere etc (rectangles in FIG. 1). See for instance WO 99058245, WO 02074438, US 20040202579, WO 2004105890, WO 2004103891 (all Gyros AB). Contact angles refer to values at the temperature of use, typically ±25° C., are static and can be measured by the method illustrated in WO 00056808 and WO 01047637 (all Gyros AB).

### Experimental Part

Assay Procedure

The invention was investigated in two model systems:
a) anti-PPV antibody as analyte and solid phase bound PPV) as affinity reactant 1 (antigen and labelled PPV as affinity reactant 2 (antigen), and
b) anti-IgG monoclonal antibody as analyte, solid phase bound IgG as affinity reactant 1 (antigen), and labelled IgG as detectable affinity reactant 2 (antigen).

PPV stands for Porcine Parvovirus. The main steps of the assay as used in the experimental part step 1) Capture reagent addition=addition of biotinylated PPV-antigen or biotinylated IgG in combination with biotinylated bovine serum albumin (BSA), step 2) Analyte addition/Capturing step=addition of sample containing analyte (anti-PPV antibody or anti IgG antibody); and step 3) Measuring/Detection step=addition of fluorophor labelled PPV antigen or fluorophor labelled IgG. The particular assay protocol selected is called Bioaffy 1C v2 and its detailed is given at the end of the experimental part.

The abbreviation “PPV” refers to an antigen preparation from porcine parvovirus.

Microfluidic Device and Instrumentation

The microfluidic device was the same as the one shown WO 04083108 (Gyros AB) and WO 04083109 (Gyros AB). The solid phase was polystyrene particles coated with phenyldextran to which streptavidin had been immobilized and packed to a bed/column in the reaction microcavity (104). The instrument used for processing was a Gyrolab Workstation equipped with laser fluorescence detector and the microfluidic disc a Bioaffy CD microlaboratory, both being products of Gyros AB, Uppsala, Sweden.

### EXAMPLE 1

### Assay of Anti-PPV Antibody

Analytes: Rabbit polyclonal anti-PPV antiserum and a mouse anti-PPV IgG monoclonal from National Veterinary Institute, Uppsala, Sweden. Mouse anti-PPV IgG monoclonal from Svanova, Sweden (Svanovir™: ELISA test for the detection of PPV antibodies in serum, Manual number 19-7400-00/04). Serum samples collected from mice during various stages of immunization with PPV antigen. Standards were pooled sera from mice during immunization with PPV diluted in steps of 5 (⅕ to 1/78125).

Reagents PPV and bovine serum albumin (BSA). PPV was obtained from Rivera, E at the National Veterinary Institute, Uppsala, Sweden (Rivera E et al., “The Rb1 fraction from ginseng elicits Th1 immunity” Vaccine (in press)). Pool II of the preparation was used.

Biotinylated Reagents (PPV=Affinity Reactant 1, BSA)
50 μg of PPV was used. In order to obtain a concentration of 1 mg/mL, 500 μL of the virus fraction was centrifuged in a Nanosep 30K filter from Pall Corporations. EZ-Link-Sulfo-NHS-LC-Biotin (Pierce) was diluted to 10 mM and used in 20 times molar excess. The solutions were mixed and incubated in room temperature for 40 minutes. Free biotin was removed by placing a volume of 450 μL PBS (0.015M NaPO₄ pH 7.4, 0.15M NaCl, 0.02% NaN₃) together with the reaction mixture on the membrane of a Nano Sep 30K column followed by centrifugation at 13,000 rpm until about 50 μL remained. To be sure that free biotin was removed another wash with 450 μL PBS was performed. The final volume was 60 μl.
Essentially the same procedure was used for biotinylation of bovine serum albumin (BSA). The starting concentration was 1 mg BSA per mL in a volume of 300 μL. The biotin reagent was used in twelve time's molar excess. The purification step was performed using protein desalting spin columns from Pierce. The final volume was 330 μL.

Fluorophor Labeled PPV (Affinity Reactant 2)

90 μg the virus preparation was concentrated to 1 mg/mL using Nanosep 30K filter from Pall Corporations and labeled with Alexa fluorophor 647 monoclonal antibody labelling kit (A-20186, Molecular Probe) according the manufacturer's instruction. The final product had a volume of 90 μL.

Titration of Immobilized Biotinylated Reactants (Immobilized PPV=Affinity Reactant 1)

In order for PPV to bind to antibodies in a detectable manner, biotinylated PPV together with biotinylated Bovine Serum Albumin (BSA) was immobilized on the solid phase. A suitable combination of concentrations had to be established so that the antibody would be able to bind both the biotinylated and the labeled reagents i.e. “bridging” the two antigen preparations. In a conventional antibody assay this measure would not be needed as long as the column is saturated with antigen. However, in the assay according to the invention the antibody is not allowed to bind with both arms to the immobilized antigen (affinity reactant 1) since this would prevent binding to the detecting antigen (affinity reactant 2). On the other hand, there must be enough antigen (affinity reactant 1) immobilized on the solid phase to generate a response, or the assay is not useful. If the reaction equilibrium between antigen and antibody were shifted too much to either side, it would in theory be impossible to obtain a signal.

Different 1:1 combinations of diluted/undiluted stock solutions of biotinylated PPV (dilutions 1/10- 1/1280) and biotinylated BSA (dilutions 1- 1/128) in PBS-T were tested. The purpose of using BSA together with PPV is to fully saturate the streptavidin column with protein and thus, avoid unspecific surface interactions between labeled PPV and the solid phase including its pre-immobilized streptavidin.

To promote bridge binding, a mixture of biotinylated BSA and biotinylated PPV were added in the capture reagent addition step. The signal ratio between blank and sample was calculated. A large ratio between sample and background response is desired for an assay with high performance. The results suggested a dilution for PPV in the interval of 80-160 times and for BSA a dilution in the interval of 16-64 times.

To study the titrations in more detail, diagrams of the viewer (Software program in the instrument used) were compared and evaluated. It could be observed that more PPV on the column led to a greater signal and to an enrichment of the analyte at the top of the column. The absolute response values revealed a turning point at a dilution of 1/80 for 1/64 biotinylated BSA. The viewer showed that more biotinylated BSA gave the peak a broader base and the profile appears to be collapsing. Column profiles with peaks at the top of the column were favored for the algorithm to integrate as much of the signal as possible.

It could be concluded that the ratio between BSA and PPV should be about 1.57. In the interest of saving reagents, the chosen dilutions were 1/100 for PPV and 1/64 for BSA mixed together as 1:1. This was later modified to 1/80 for PPV and 1/51 for the BSA preparation.

Titration of Fluorophor Labeled PPV (Affinity Reactant 2)

The optimal concentration for fluorophor labeled reactant was also tested by using the stock solution of fluorophor labeled PPV in three different dilutions, 1/10, 1/20 and 1/40 in PBS with 1% BSA. Rabbit anti-PPV serum was serially diluted and used as a reference sample to generate data points for all titrations. It was found that the dilution 1/40 gave the lowest background. This dilution was consequently used.

Performance

Precision: The mouse serum was diluted 125 times in PBS with 1% BSA and aspirated in twelve repeats and assayed. The coefficient of variance (CV) was 1.89%.

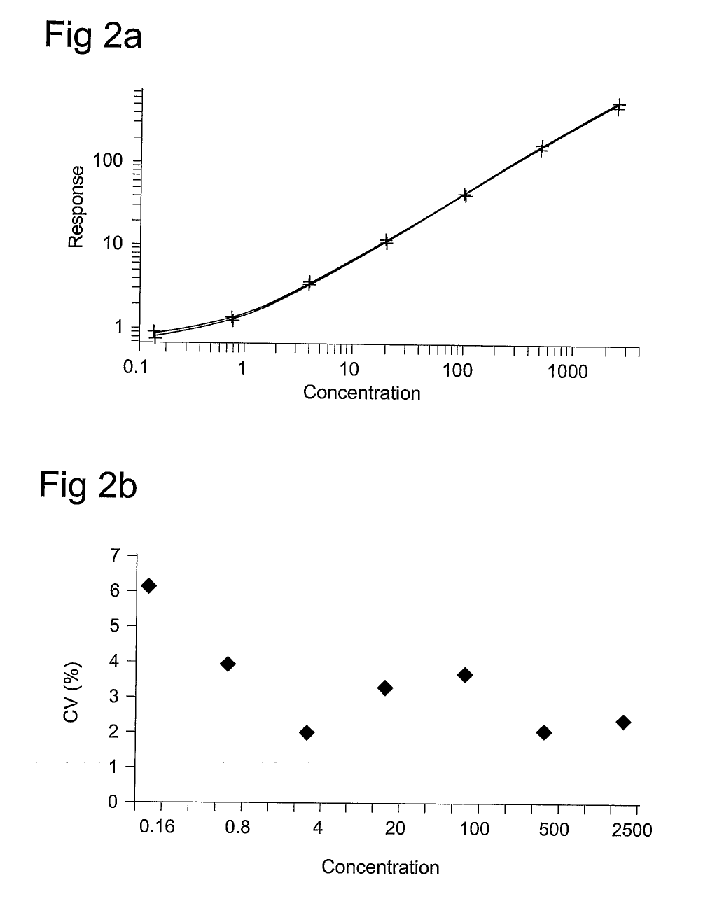

Measuring range: See FIG. 2.

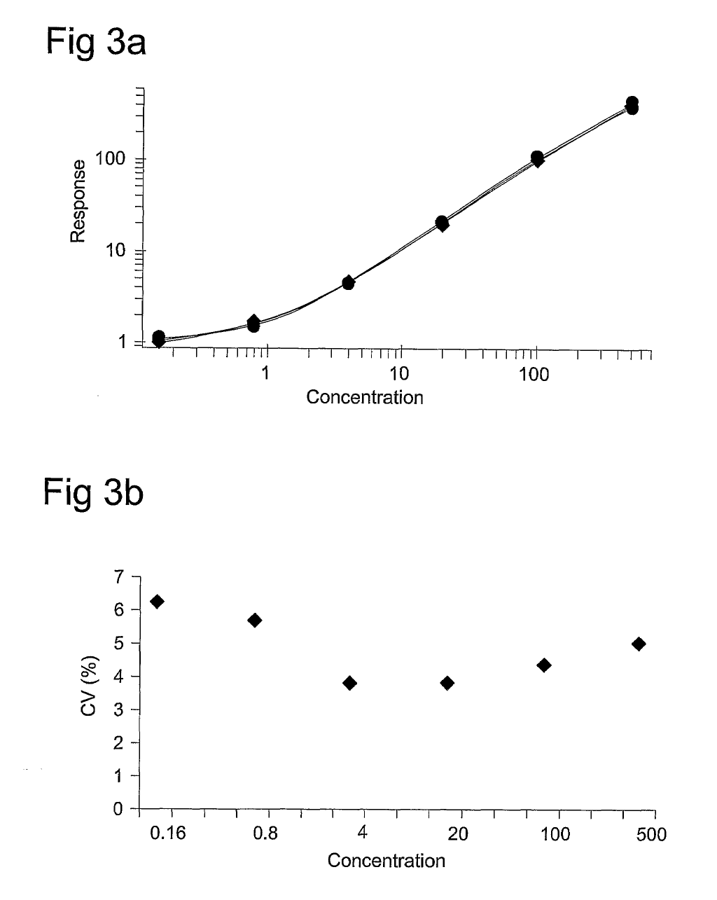

Reproducibility: See FIG. 3.

Sample Dilution

To evaluate possible dilution factors for serum samples, two mice from with low titers of anti-PPV, two mice with intermediate titers of anti-PPV and two mice with high titers of anti-PPV were selected. All samples were taken two weeks after immunization. The samples were diluted as ½, ¼, ⅛, 1/16 and 1/32 and run in triplicates. The samples were analyzed at different dilutions without any technical difficulties. The least diluted samples could be measured for all mice and could be distinguished from the background signal.

Gel Filtration

In order to prove that the bridging assay is independent of immunoglobulin class, a gel filtration experiment was performed. Sera was taken at two occasions from five mice during PPV immunization and pooled to 100 μl: a) two weeks after immunization (2vI) where IgM could be expected and b) five weeks after booster (5vII) where mostly IgG is present. The pools were separately chromatographed on Superdex™ (ÄKTA FPLC™) by first washing the column three times with milliQ water followed by two equilibrations with degassed PBS whereafter the serum sample was injected. Fractions were collected in microtiter wells. Every second fraction within the detection range was tested in the inventive assay method on Bioaffy™ CD microlaboratory.

Activity was found at two distinct peaks for the pool obtained two weeks after immunization. The lower activity peak was close to the void volume while the higher activity peak was close to the albumin retention volume. Hence, the molecular size of the analyte varied widely and it seemed likely that PPV specific IgM contributed to the low activity peak and PPV specific IgG to the high activity peak. For the sample taken five weeks after booster only one activity peak could be seen. This peak was mapped to the area in the chromatogram where IgG would be expected.

Quantification of unknown samples. A total of 234 sera from mice during immunization with PPV were run in triplicates at a normal dilution of 1/25. This dilution proved to be insufficient for samples with high anti-PPV antibody titers for which saturated columns were found. Samples displaying saturated columns were rerun at higher dilutions.

### EXAMPLE 2

### Assay of Anti-IgG Antibody

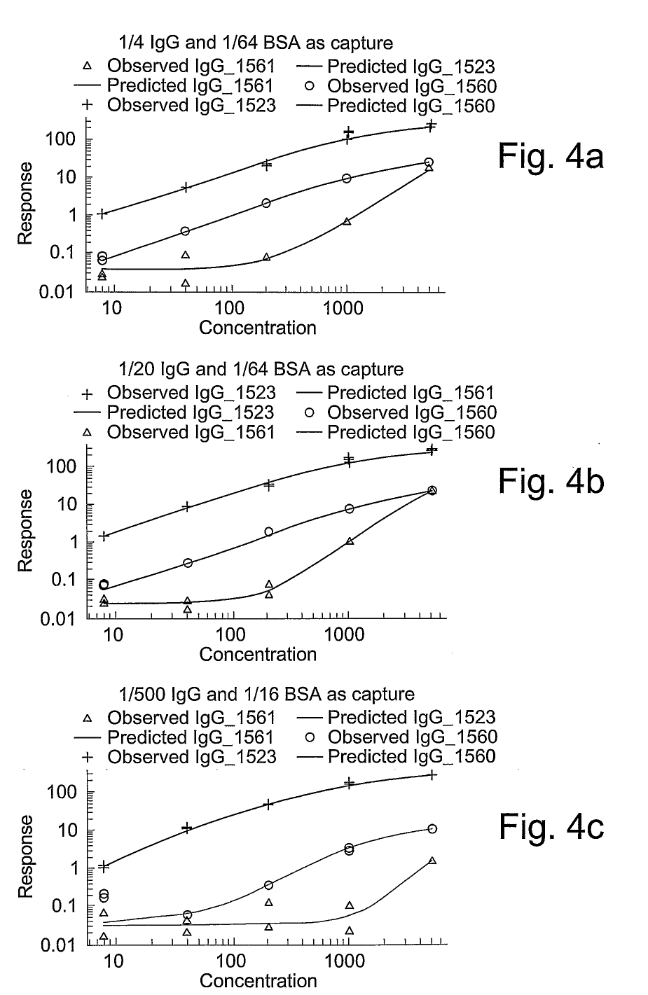

Analytes: One polyclonal antibody from Sigma (prod. nr 555784) and three monoclonal antibodies specific for human IgG were used as analytes, where BD Pharmingen supplied one clone (I9885) and Fitzgerald supplied the remaining two clones (10-I21 and 10-I17). The monoclonals are called 1561, 1523 and 1560 in FIG. 4a-c.

Reagents

Human monoclonal IgG1λ from a myeloma (Sigma) was used as antigen (hIgG)
hIgG was labeled with biotin in the same manner as described above for PPV. For the biotin reaction, the first step was to exchange buffers since Tris in the storage buffer could interfere with the biotin reagent. Nanosep 30K filters from Pall Corporations were used for this purpose. After removal of Tris, EZ-Link-Sulfo-NHS-LC-Biotin (Pierce) was diluted to 10 mM and added in 12 times molar excess to 100 μL of hIgG solution. The solutions were mixed and incubated in room temperature for 1 h. Absorbance at 280 nm was measure and the protein concentration was determined to 3.45 μM. Biotinylated hIgG=affinity reactant 1)
Fluorophor labeling was carried out as described for PPV above. The starting amount of hIgG was 100 μg. The degree of labeling was determined by measuring absorbance at 280 nm and at 650 nm to about 7 moles ALEXA (fluorophor) per mole protein with a final concentration of 2.76 μM. Fluorophor labeled hIgG=affinity reactant 2.

Titrations of Reagents

The biotinylated reagents (hIgG and BSA) were titrated to find the optimal dilutions for the assay system before different monoclonal antibodies were tested as analytes. The procedure was essentially the same procedure as for PPV, with different dilutions of the stock solutions of biotinylated hIgG mixed in a 1:1 ratio with biotinylated BSA. The response ratio between signal and blank together with the column profiles formed the basis for the final selection of the most favorable combination. The combination 1/100 of biotinylated hIgG and 1/64 of biotinylated BSA was chosen.

Dilutions of the detecting antigen (fluorophor labeled hIgG) were also tested to obtain a large signal to noise ratio. Flurophor-labeled hIgG was run with dilutions factors of 20, 40 and 80. A dilution factor of 40 was selected.

For all, titration experiments a polyclonal antibody was used as analyte reference.

Different Amounts of Biotinylated Antigen

Three monoclonal mouse anti-human IgGs were tested with a combination of biotinylated hIgG (B*hIgG) together with biotinylated BSA in a 1:1 ratio (see below).

| | |
|---|---|
| ¼ B * hIgG | with 1/64 B * BSA |
| 1/20 B * hIgG | with 1/64 B * BSA |
| 1/100 B * hIgG | with 1/64 B * BSA |
| 1/500 B * hIgG | with 1/16 B * BSA |

To ensure that the streptavidin column is saturated with protein and to avoid unspecific interactions, a dilution of 1/16 BSA was used with the most diluted biotinylated IgG. The monoclonal anti-hIgG antibodies (analytes) were separately run with concentrations ranging from 5000 ng/mL down to 8 ng/mL generating curves from five data points. Detection was done with 69 nM fluorophor labeled hIgG.

The column profiles studied in Gyrolab viewer show higher response levels with more antigen (hIgG) in the capture reagent mixture than when less antigen is added to the column. The peaks of all three antibodies seem to collapse when less biotinylated antigen is present and there is a tendency for more plateau-like peaks as well. It actually seemed to be possible that by including less antigen in the capture reagent, antibodies with low affinity to the antigen will fade away and eventually approach blank levels. This possibility is also supported by the standard curves obtained for the three monoclonal anti-IgG antibodies used as analytes. See FIG. 4a-c.

The assay protocol used (Bioaffy 1C 2v) comprises the steps:
Initial needle wash: Particle wash 1, Particle wash spin 1, Particle wash 2, and Particle wash spin 2
Capture reagent addition: Capture reagent spin, Capture reagent wash 1, Capture reagent wash spin 1, Capture reagent wash 2, Capture reagent wash spin 2
Analyte addition: Analyte spin, Analyte wash 1, Analyte wash spin 1, Analyte wash 2, and Analyte wash spin 2
CD alignment 1: Detect background PMT 1%, Detect background PMT 5% and Detect background PMT 25%, Spin out, Detection reagent addition, Detection reagent spin, Detection reagent wash 1, Detection reagent wash spin 1, Detection reagent wash 2, Detection reagent wash spin 2, Detection reagent wash 3, Detection reagent wash spin 3, Detection reagent wash 4, Detection reagent wash spin 4
CD alignment 2: Detect PMT 1%, Detect PMT 5%, Detect PMT 25%

### EXAMPLE 3

### Assay of Heterophilic Antibodies

This assay was designed after that significant positive assay responses had unexpectedly been found in several serum and plasma samples (citrate plasma, heparin plasma, EDTA plasma) by a sandwich hTNFα assay utilizing R-1530 mouse anti-hTNFα antibody as capture antibody and another mouse anti-hTNFα antibody as detector antibody.

Microfluidic device and instrumentation: The same as for examples 1 and 2.

Assay procedure: As outlined for examples 1 and 2.

Samples: Sera from 16 blood donors. The sera were thawed in refrigerator during night, vortexed and finally centrifuged for 15 min at 4000 rpm in Eppendorf centrifuge at +8° C.

Buffers

PBS-T: 15 mM PBS pH 7.4, Tween™ 0.05%, NaN₃ 0.02%

PBS-BSA: 15 mM PBS pH 7.4, NaN₃ 0.02%, 1% BSA

Reagents:

| Capture Ab¹⁾ (affinity reactant 1) | Detecting Ab²⁾ (affinity reactant 2) |
|---|---|
| R-1530 mouse anti-hTNFα | F-1197 mouse anti-hTNFα (IgG1) |
| 0.1 mg/mL³⁾ | 25 nM³⁾ |
| | F-1181 goat anti-hIL-1β (polyclonal) |
| | 50 nM³⁾ |
| | F-1210 rat anti-hIL-5 (IgG2A) |
| | 50 nM³⁾ |
| | F-1155 mouse anti-hTNFβ (IgG1) |
| | 50 nM³⁾ |

¹⁾Biotinylated as outlined for hIgG in example 2
²⁾Labelled with Alexa 647 as outlined for hIgG in example 2.
³⁾Concentrations of solutions introduced into the microfluidic device

Reference Standard:

Recombinant hTNFα in the interval 2.4-1750 pg/mL in PBS-BSA.

Results:

Significantly elevated levels of heterophilic antibodies could be measured in several of the serum samples. In principle the same relative variation could be obtained for each detecting antibody and for EDTA-, heparin- and citrate-plasma corresponding to the blood donor sera.

Certain innovative aspects of the invention are defined in more detail in the appending claims. Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for the determination of an analyte which is a) present in a liquid sample 1 suspected of containing the analyte, and b) at least bivalent with respect to ability to simultaneous affinity bind at least two 5 equal binding structures BSs, by the formation of an affinity complex that comprises the analyte and an affinity reactant 1 that exhibits the BS and is immobilized to a solid phase, characterized in comprising the steps of:
(i) providing a flow path in the form of a microchannel structure of a microfluidic 0 device, said structure comprising a reaction cavity in which there is a solid phase to which affinity reactant 1 is immobilized, (ii) providing sample 1 at a position upstream of the cavity and flowing sample 1 through the reaction cavity for the formation of the affinity complex under flow conditions, 5 (iii) measuring the amount of complex formed in the solid phase by a. providing a liquid sample 2 that contains an analytically detectable and soluble affinity reactant 2 that comprises a binding structure BS in a position upstream of the reaction cavity, b. flowing this sample 2 through the cavity after sample 1 has passed the same 0 cavity, and c. measuring the amount of affinity reactant 2 incorporated into the complex.

2. The method of claim 1, characterized in that the analyte is an anti-BS antibody.

3. The method according to claim 2, characterized in that the anti-BS antibody comprises subpopulations of anti-BS antibodies differing in affinity for BS.

4. The method according to claim 2, characterized in that the anti-BS antibody is a mixture of different monoclonal anti-BS antibodies.

5. The method according to claim 2, characterized in that the anti-BS antibody is a polyclonal anti-BS antibody, i.e. is a native anti-BS antibody preparation containing a mixture of monoclonal anti-BS antibodies deriving from different cells.

6. The method according to claim 2, characterized in that the density and amount of BS in the solid phase and the flow rate during step (ii) has been adapted to each other so that only a fraction of the antibody subpopulations are captured by BS of the solid phase.

7. The method according to claim 1, characterized in that the BS of the solid phase is attached to the solid phase by the use of an immobilizing pair of reactive structures that are mutually reactive with each other to the formation of a bond that resist processing during the method, one of said reactive structures (RSₛₚ) is pre-10 introduced on the solid phase before step (i) while the other reactive structure (RSₐᵣi) is present on affinity reactant 1 and said formation is carried out either before or during step (i).

8. The method according to claim 7, characterized in that said immobilizing pair is an 15 immobilizing affinity pair comprising a ligand L that is attached to the solid phase and the counterpart is an immobilizing binder B that is linked to BS.

9. The method according to claim 8, characterized in that the solid phase used in step (ii) comprises structures that derive from reactive RSₛₚ structures that have not been utilized for the immobilization of BS.

10. The method according to claim 1, characterized in that said solid phase is in the form of a porous bed.

11. The method according to claim 1, characterized in that a) said analyte is an antibody, b) said sample derives from a body fluid of an animal after exposure or after a suspected exposure to an antigen comprising BS, and c) said method is performed in order to determine whether or not said exposure has 30 occurred or the status of an immune response raised in said animal upon said exposure.

12. The method according to claim 1, wherein the method is used for the diagnosis of a disease related to said analyte.
